(19) United States
(12) Patent Application Publication
(10) Pub. No.: US 2021/0042678 A1
(43) Pub. Date: Feb. 11, 2021

(54) DECISION SUPPORT SYSTEM FOR HOSPITAL QUALITY ASSESSMENT

(71) Applicant: BATTELLE MEMORIAL INSTITUTE, Columbus, OH (US)

(72) Inventors: Jeffrey J. Geppert, Columbus, OH (US); Michele Morara, Miami, FL (US); Warren Strauss, Lewis Center, OH (US)

(21) Appl. No.: 17/077,619

(22) Filed: Oct. 22, 2020

Related U.S. Application Data
(63) Continuation of application No. 15/307,821, filed on Oct. 29, 2016.
(60) Provisional application No. 61/986,134, filed on Apr. 30, 2014.

(51) Int. Cl. G06Q 10/06; G16H 50/20; G16H 40/20

(52) U.S. Cl. CPC G06Q 10/06393; G16H 40/20; G06Q 10/06; G16H 50/20

(57) ABSTRACT

A decision support system comprises receiving a request from a client computer to derive a quality assessment associated with a health care provider of interest, receiving an identification of a user-selected benchmark, determining a comparison range over which data from the data source is to be analyzed, identifying a set of quality measures, generating a first data set of quality measure performance by evaluating the set of quality measures against underlying medical data in a data source filtered by the range, generating a second data set defining an estimated quality measure performance using a probabilistic forecasting model to evaluate the set of quality measures by drawing inferences about the set of quality measures beyond a period of time for which the underlying medical data is available. An overall quality indicator score is computed, based upon a comparison of the first data set and the second data set.

### CROSS REFERENCE TO RELATED APPLICATIONS

This application is a continuation of U.S. patent application Ser. No. 15/307,821, filed Oct. 29, 2016, entitled “DECISION SUPPORT SYSTEM FOR HOSPITAL QUALITY ASSESSMENT”, which is a § 371 National Stage of International Application No. PCT/US2015/028229, filed Apr. 29, 2015, entitled “DECISION SUPPORT SYSTEM FOR HOSPITAL QUALITY ASSESSMENT”, which claims the benefit of U.S. Provisional Patent Application Ser. No. 61/986,134, filed Apr. 30, 2014, entitled “DECISION SUPPORT SYSTEM FOR HOSPITAL QUALITY ASSESSMENT”, the disclosures of which are hereby incorporated by reference.

### BACKGROUND

The present disclosure relates in general to hospital assessment and in particular, to decision support systems for hospital quality assessment and improvement.

Hospitals provide diagnosis, treatment, and therapy to sick and injured individuals. In this regard, many clinical health care decisions must be made in the typical course of treating a patient who is undergoing medical care. Accordingly, it is likely that the quality of service that a patient receives will vary across hospitals because these health care decisions are made by providers that possess varying levels of skill, experience, resources, etc.

### BRIEF SUMMARY

According to aspects of the present disclosure, a method is provided, for computing reference and benchmark data for evaluating healthcare providers. The method is implemented as a machine-executable process, and comprises obtaining at least two data sets including a first data set and a second data set. A third data set may also be obtained, such as where the reference and benchmark data are to be used to compute a preventability score, as will be described in greater detail herein. In this regard, the first data set (e.g., a state inpatient database) includes “present on admission” data that represents a condition of a patient that is present at the time an order for inpatient admission occurs. The second data set (e.g., nationwide inpatient sample) does not require present on admission data. The third data set (e.g., hospital association data) also includes present on admission data. The method further comprises establishing quality measures including obtaining a set of quality indicators (e.g., quality indicators identified by the Agency for Healthcare Research and Quality), and evaluating each of the first data set, the second data set and optionally, the third data set against the obtained quality indicators.

The method still further comprises calibrating, by a processor, the expected present on admission data of the first data set as a Recalibration Factor such that an overall observed rate (P) equals an overall expected rate (E[P|X]) for each measure of the first data set. Also, the method comprises using, by the processor, the Recalibration Factor to calculate expected present on admission data on the second data set, and using the calculated expected present on admission data of the second data set to calculate an observed and expected outcome of interest on the second data set. The method yet further comprises using the calculated observed and expected outcome of interest of the second data set to forecast an observed and expected outcome of interest for the second data set. Moreover, the method comprises using the calculated observed and expected outcome of interest on the second data set and the forecasted observed and expected outcome of interest on the second data set to calculate an overall observed-to-expected ratio and a reference population rate (K) for each measure of the second data set, and using a predetermined signal variance (e.g., from software provided by the Agency for Healthcare Research and Quality) and the reference population rate on the second data set to calculate a national benchmark for each measure.

According to further aspects of the present invention, the method further comprises computing a preventability score that characterizes a proportion of adverse events that were potentially preventable in accessing an healthcare provider of interest. The preventability score is computed by obtaining reference and benchmark data, and using the calculated expected outcome of interest on the second data set and the forecasted expected outcome of interest on the second data set to calculate an expected outcome of interest on the third data set. The preventability score is further computed by using an observed outcome of interest on the third data set, a calculated expected outcome of interest on the third data set, and the reference population rate from the second data set to calculate a risk-adjusted rate on the third data set and a noise variance on the third data set, for each measure in the third data set. The preventability score is still further computed by using the risk-adjusted rate on the third data set, the noise variance on the third data set and a predetermined signal variance to calculate a performance score on the third data set and a “posterior variance” on the performance score on the third data set for each measure.

According to further aspects of the present invention, a decision support system is implemented by a computer system that comprises a processing device and a server that are linked together by a network, where the network is supported by networking components. The server executes a processing engine that interacts with at least one data source, wherein the processing engine is implemented by a computer program product embodied in one or more computer readable storage medium(s) (storage hardware) having computer program instructions embodied thereon, such that the instructions execute via a processor of the server to receive a request from a client computer to derive a quality assessment associated with a health care provider of interest, where the quality assessment populates a dashboard on the client computer. The computer program instructions also receive identification of a benchmark that is associated with the quality indicator, where the benchmark defines at least one entity to compare against the health care provider of interest. Here, the benchmark may be computed by the computer program instructions, e.g., as set out more fully herein.

The computer program instructions also determine a comparison range over which data from the data source is to be analyzed for deriving the quality indicator, identify a set of quality measures that each assesses a different aspect of health care, and generate a first set of evaluations by evaluating the set of quality measures against a subset of the underlying medical data in the data source that has been filtered by the range.

The computer program instructions further generate a second set of evaluations defining an estimated quality measure performance using a probabilistic forecasting model to evaluate the set of quality measures for the healthcare provider of interest, where the second data set draws inferences about the set of quality measures beyond a period of time for which the underlying medical data is available to the data source for the healthcare provider of interest. The computer program instructions still further compute a single, overall quality indicator score, based upon a comparison of the first data set, the second data set, and the benchmark, and communicate the computed overall quality indicator score for visual representation in the dashboard on the client computer.

### BRIEF DESCRIPTION OF THE SEVERAL VIEWS OF THE DRAWINGS

FIG. 1 is a block diagram of a basic computer system that may be used to implement a decision support system, according to aspects of the present disclosure;

FIG. 2 is a method of computing a national reference and benchmark, according to aspects of the present disclosure herein;

FIG. 3 is a method of computing a preventability score, according to aspects of the present invention;

FIG. 4 is a method of establishing quality indicators for use with the method of FIG. 2, according to aspects of the present disclosure;

FIG. 5 is a flow chart of a process for computing an overall quality indicator, according to aspects of the present disclosure;

FIG. 6 is a screen shot of an exemplary Entry screen for a decision support dashboard according to aspects of the present disclosure;

FIG. 7 is a screen shot of an exemplary Summary screen for a decision support dashboard according to aspects of the present disclosure;

FIG. 8 is a screen shot of an exemplary explanation for a performance measure within the decision support dashboard;

FIG. 9 is a screen shot of an exemplary Detail screen for a user-selected performance measure of the decision support dashboard according to aspects of the present disclosure; and

FIG. 10 is a block diagram of a computer system for implementing the systems and methods described herein.

### DETAILED DESCRIPTION

According to various aspects of the present disclosure, systems, methods and computer program products implement decision support systems for health care provider quality assessment and improvement. In this regard, aspects herein disclose the creation of national reference and benchmark data that account for present on admission conditions. The national reference and benchmark further align with the most currently available data from hospital associations. Moreover, aspects of the present disclosure herein compute a “preventability score” that defines a proportion of adverse events that were potentially preventable.

Aspects of the present disclosure also provide navigable dashboard displays that enable a user to explore computed measures that are indicative of the quality of a health care provider of interest, compared to a corresponding national average or other benchmark groupings of health care providers. In illustrative implementations, the computed measures are stratified by predefined quality measures. For instance, the dashboard may be utilized to provide health care providers with data such as trends over time for a composite quality measure (across all conditions), a single metric associated with a composite overall quality performance placed on a 0-1000 score, and an empirical distribution of this composite score across a user-selected benchmark grouping of health care providers, etc.

Further aspects of the present disclosure provide a simulation tool that allows health care providers to estimate the number of anticipated adverse events over a defined period of time (e.g. calendar year 2014) associated with a particular quality measure based on their current trends. The simulation tool may be useful for instance, to estimate the amount of money at-risk from a reimbursement perspective associated with that number of adverse events, estimate of the amount of additional money that would be either gained or lost if the number of adverse events changes from the estimated value, etc.

Platform Overview:

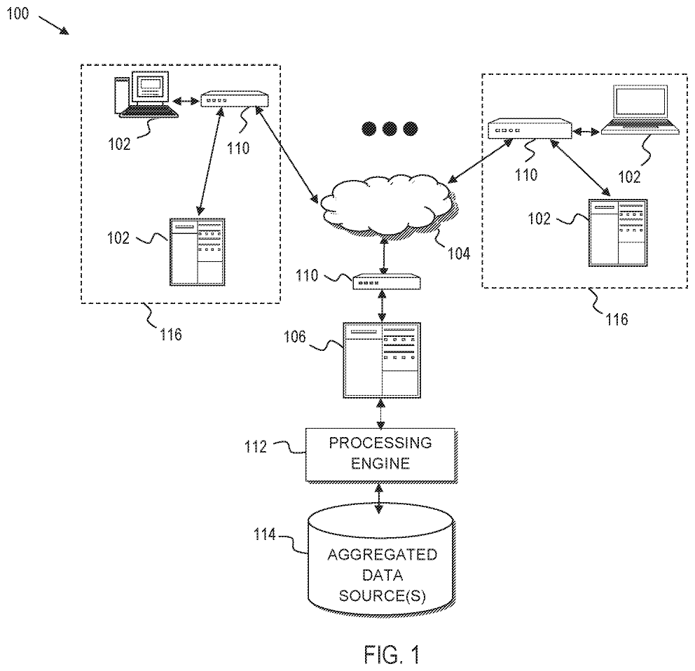

Referring now to the drawings and particularly to FIG. 1, a general diagram of a computer system 100 is illustrated, where components of the computer system 100 can be used to implement elements of a decision support system according to aspects of the present disclosure. The computer system 100 is implemented as a distributed system that facilitates the interaction of multiple entities, e.g., hospitals, data aggregators, national and state-level database collection resources, third party providers, etc. However, the computer system 100 may be implemented on a relatively smaller scale, within a hospital, clinic or other health care facility. Likewise, the computer system 100 can be expanded out to include one or more intermediates that participate in the decision support system.

The computer system 100 comprises a plurality of processing devices 102 that are linked together by a network 104 to a decision support server 106. As will be described more fully herein, some processing devices 102 of the computer system 100 are used to execute a corresponding decision support application, e.g., a user interface such as a decision support dashboard. In other exemplary implementations, a processing device 102 may be utilized by a health care provider to upload medical data, e.g., administrative data extracted from a local data source, for processing and analysis by the decision support server 106. Still further, some processing devices 102 may provide a source of data, such as for quality measures, quality indicators, data set(s), or other information used by the decision support system as set out in greater detail herein.

As a few illustrative examples, the processing devices 102 can include servers, personal computers, portable computers, etc. As used herein, portable computers include a broad range of processing devices, including notebook computers, netbook computers, tablet computers, personal data assistant (PDA) processors, cellular devices including Smartphone and/or other devices capable of communicating over the network 104.

The network 104 provides communications links between the various processing devices 102 and the decision support server 106, and may be supported by networking components 110 that interconnect the processing devices 102 to the decision support server 106, including for example, routers, hubs, firewalls, network interfaces, wired or wireless communications links and corresponding interconnections, cellular stations and corresponding cellular conversion technologies, e.g., to convert between cellular and tcp/ip, etc. Moreover, the network 104 may comprise connections using one or more intranets, extranets, local area networks (LAN), wide area networks (WAN), wireless networks (WIFI), the Internet, including the World Wide Web, and/or other arrangements for enabling communication.

The decision support server 106 executes at least one processing engine 112 that interacts with aggregated data sources 114 to execute the methods herein. For instance, as will be described in greater detail herein, the decision support server 106, e.g., via the processing engine 112, performs analyses to compare the quality of health care providers, such as hospitals, against benchmarks (e.g., a national average, state average, the hospital's own past performance, etc.). The quality computations are stratified by quality measure, and can be used to predict future trends for quality and risk.

For instance, the processing engine 112 may execute a model or set of models (e.g., based upon the national Quality Indicator models, nationally representative administrative data and optionally, other available data) to evaluate healthcare performance. The processing engine 112 may also utilize probabilistic forecasting models to extend inferences beyond the period of time for which models and administrative data are available. As such, the system herein closes the temporal gap between available data and time periods of interest to users in evaluating health care provider quality.

Also as will be described in greater detail herein, the aggregated data sources 114 comprise different data sources that are processed and analyzed to facilitate the decision support as described more fully herein. For instance, the various data sources may be obtained from one or more of the processing devices 102, and may include data collected from national, state, regional, local, (or combinations thereof) data aggregators, national Quality Indicator models, nationally representative administrative data, etc.

As illustrated, multiple independent entities 116 can interact with the decision support server 106. In this regard, an entity 116 may be a health care provider, e.g., a hospital, clinic, treatment center, etc. In this regard, the entity may be one location or a distributed system, e.g., with multiple locations. Moreover, an entity 116 may include an association or hospital membership organization that manages a number of health care providers. Still further, an entity 116 may be a data aggregator that shares data with the decision support server 106.

Many current hospital quality measures and quality indicator models are based on patient level administrative data, e.g., patient discharge records. This patient level administrative data may be communicated, e.g., via a processing device 102, from the local data of a corresponding health care provider to the aggregated data sources 114. The local data may also store hospital level information, which is communicated to the aggregated data sources.

Further, data stored in the aggregated data sources 114 and which is displayed through the software dashboard herein, may be largely based on administrative billing records that participating hospitals already submit to the Federal Government through the Healthcare Cost and Utilization Project (HCUP), thereby reducing burden to hospitals in data delivery to the decision support server 106 to make use of the dashboard tool.

Entities 116, such as hospitals, hospital systems, and hospital membership organizations may also provide the decision support server 106 with access to their administrative data in the same format that they utilize for HCUP submissions on a quarterly basis. The decision support server 106 can thus conduct statistical and economic modeling of these data resources utilizing a system of programs implemented in a Health Insurance Portability and Accountability Act (HIPAA) compliant data center, e.g., as executed on the decision support server 106 and then display the results of these analyses in a series of dashboard tools that will be delivered through a secure website over the Internet (network 104) to a client computer, e.g., processing device 102. The decision support server 106 may also work with hospitals and hospital systems to capture other data from electronic health records or other available data sources (under a consistent data format) to extend the utility of the quality measures beyond administrative data.

The flows, methods, processes, systems, etc., described with reference any of subsequent FIGURES herein can be implemented on one or more of the system components of FIG. 1, e.g., the processing engine 112 executing on the decision support server 106 interacting with the aggregated data sources 114. Moreover, the flows, methods, processes, systems, etc., described with reference any of subsequent FIGURES herein can be implemented as methods or computer program product code that is embodied on a computer readable storage media (computer-readable hardware). The code is executable by a processor to cause the processor to perform the corresponding methods set out herein.

Decision Support System:

According to aspects of the present invention, a decision support system is constructed through the acquisition of healthcare related data sources, which are utilized in the creation of national reference and benchmark data that account for present on admission data. The national reference and benchmark data is ultimately utilized in the computation of a “preventability score” that is displayed in a dashboard view, as will be described in greater detail below.

Reference and Benchmark Data:

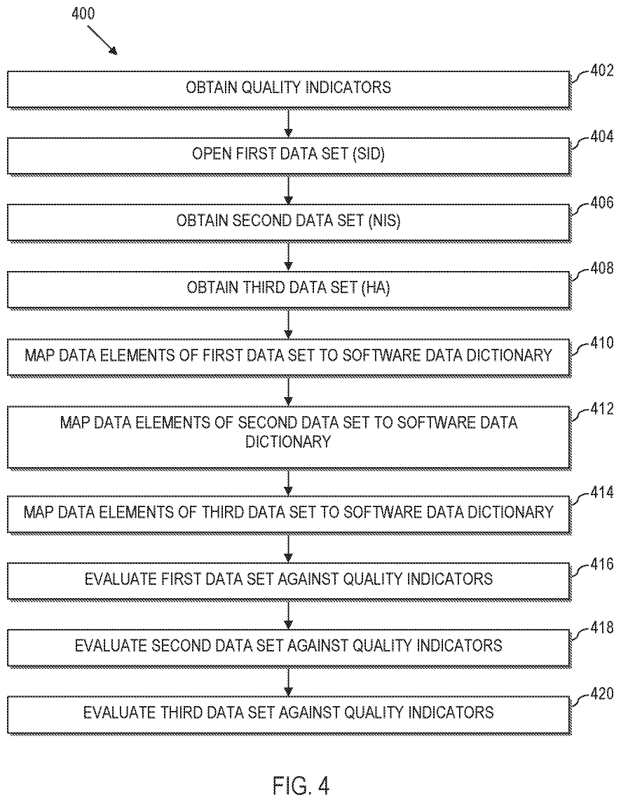

A method is provided for computing reference and benchmark data for evaluating healthcare providers. The method 200 comprises establishing at 202, quality measures, e.g., for at least three sample data sets. An example method of establishing the quality measures is discussed in greater detail with reference to FIG. 4. However, in general, the establishment of the quality measures at 202 includes three activities, including obtaining data sets (e.g., at least three data sets), obtaining a set of quality indicators, and evaluating the data sets against the obtained quality indicators.

The first data set, e.g., a state-wide data set, should include “present on admission” (POA) data. POA data represents a condition of a patient that is present at the time an order for inpatient admission occurs. For instance, a person may have a broken arm, but is admitted because of a heart attack. The broken arm of the patient was not a result of patient care provided by the healthcare provider, and is thus considered present on admission data. As another example, conditions that develop during an outpatient encounter, including emergency department, observation, or outpatient surgery, are considered POA.

The second data set, e.g., a national data set, does not require POA data. In certain examples, the second data set does not have POA data. The third data set may be obtained from a hospital association. The third data set should include POA data. Moreover, in practical implementations, the first and second data sets may overlap in date range of included data. However, the third data set is likely to encompass data across a date range that is more recent than the data included in the first and second data sets.

The quality measures represent measures that can be used to highlight potential quality concerns, identify areas that need further study and investigation, and track changes over time. In an illustrative example, the measures comprise quality indicators from the Agency for Healthcare Research and Quality, such as Inpatient Quality Indicators (IQI), Patient Safety Indicators (PSI) and Pediatric Quality Indicators (PDI).

In summary, the method 200 applies the above-quality indicators against the first data set to calculate an observed present on admission (P) value for each discharge and measure. The method 200 also calculates an expected present on admission (E[P|X]) for each discharge and measure of the first data set. Likewise, the method 200 applies the above-quality indicators against the second data set to calculate an observed outcome of interest (Y) for each discharge and measure. The method 200 also calculates for the second data set, an expected outcome of interest (E[Y|X]) for each discharge and measure, and calculates an expected present on admission (E[P|X]) for each discharge and measure. The method 200 also applies the above-quality indicators against the third data set to calculate an observed outcome of interest (Y) for each discharge and measure. The method 200 also calculates for the third data set, an observed present on admission (P) value for each discharge and measure, and calculates an expected outcome of interest (E[Y|X]) for each discharge and measure.

More particularly, the method 200 calibrates at 204, the expected present on admission data of the first data set (e.g., the state-wide data set) as a “Recalibration Factor”. In this manner, the overall observed rate (P) equals the overall expected rate (E[P|X]) for each measure of the first data set.

The method 200 uses at 206, the Recalibration Factor (determined at 204) to calculate the expected present on admission data on the second data set (e.g., national data set). In this manner, the method calculates the expected present on admission (E[P|X]) for each discharge and measure of the second data set.

The method 200 uses at 208, the calculated expected present on admission data of the second data set (determined at 206) to calculate an observed and expected outcome of interest on the second data set. For instance, the method at 208 calculates the observed outcome of interest (Y, P=0) for each discharge and measure. The method at 208 also calculates the expected outcome of interest (E[Y, P=0|X]) for each discharge and measure of the second data set.

The method 200 uses at 210, the calculated observed and expected outcome of interest of the second data set (determined at 208) to forecast the observed and expected outcome of interest. For example, in an illustrative implementation, the method 200 forecasts the observed and expected outcome of interest at 210 using a linear trend of the observed-to-expected ratio for each healthcare provider (e.g., hospital) with a seasonally (e.g., quarterly) or other periodic effect.

The method 200 uses at 212, the calculated observed and expected outcome of interest on the second data set (determined at 208), and the forecasted observed and expected outcome of interest on the second data set (determined at 210), to calculate an overall observed-to-expected ratio and a reference population rate (K) for each measure of the second data set.

The method 200 then uses at 214, a predetermined signal variance (e.g., as may be obtained from software such as Version 4.5 SAS software provided by the Agency for Healthcare Research and Quality or as obtained in any other suitable manner) and the reference population rate on the second data set (determined at 212) to calculate a national benchmark for each measure. In an illustrative implementation, the national benchmark is specified as a percentile in a performance score distribution, e.g., 80th percentile. However, in practice, other percentiles, or other specifications may be utilized.

Preventability Score:

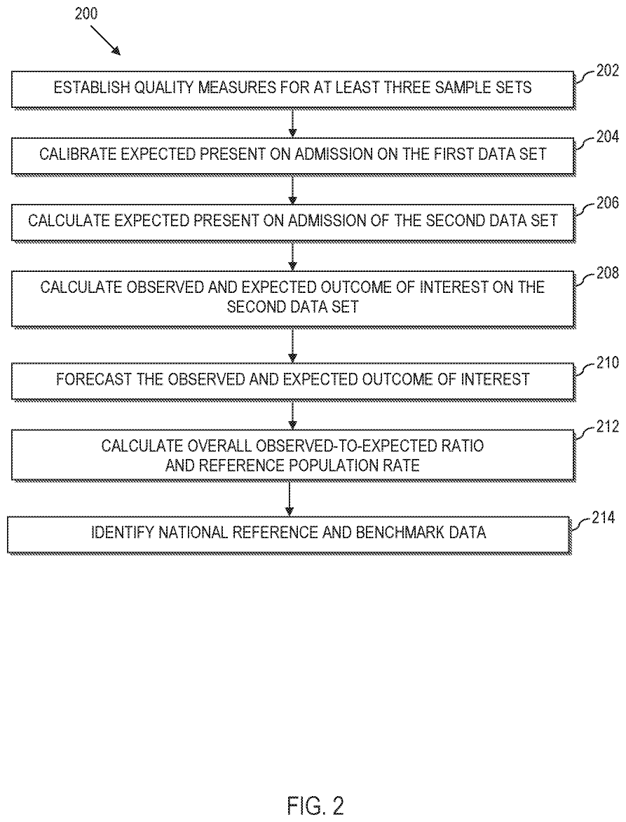

A method 300 is provided for computing a preventability score that characterizes a proportion of adverse events that were potentially preventable in accessing a healthcare provider of interest. The method 300 obtains at 302, reference and benchmark data. For instance, the method 300 may obtain the reference and benchmark data computed at 212 and 214 of FIG. 2.

The method 300 uses at 304, a calculated expected outcome of interest on the second data set (e.g., as computed at 208 of FIG. 2) and a forecasted expected outcome of interest on the second data set (e.g., as computed at 210 of FIG. 2) to calculate an expected outcome of interest on the third data set. In an illustrative example, the method 300 calculates the expected outcome of interest (E[Y, P=0|X]) for each discharge and measure of the third data set.

The method 300 also uses at 306, an observed outcome of interest on the third data set, a calculated expected outcome of interest on the third data set, and the reference population rate from the second data set (e.g., as determined at 212 of FIG. 2) to calculate a risk-adjusted rate on the third data set and a noise variance on the third data set, for each measure in the third data set.

For example, in an illustrative example, the method computes a risk-adjusted rate on the third data set as the (observed rate on the third data set/expected rate on the third data set)*reference population rate on second data set. A noise variance on the third data set is computed as a Variance (risk-adjusted rate on the third data set).

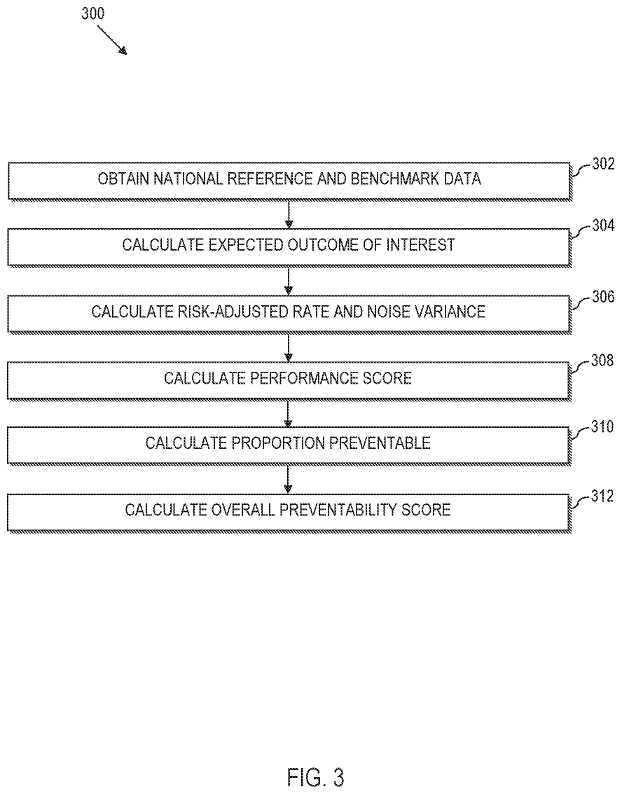

The method 300 uses at 308, the risk-adjusted rate on the third data set (determined at 306), the noise variance on the third data set (determined at 306 of FIG. 3) and a predetermined signal variance (e.g., the same predetermined signal variance determined at 214 of FIG. 2) to calculate a performance score on the third data set and a “posterior variance” on the performance score on the third data set for each measure.

In an exemplary implementation, reliability-weight (W) is computed as a (signal variance/(noise variance on the third data set+signal variance)). A performance score is computed as a risk-adjusted rate on third data set*W+reference population rate on the second dataset*(1−W). A posterior variance is computed as a signal variance*(1−W).

The method 300 uses at 310, the national benchmark (302; 214 of FIG. 2), the performance score on the third data set (determined at 308), and a posterior variance on the performance score of the third data set (308) to calculate a “proportion preventable” on the third data set for each measure. In this manner, a posterior distribution may be determined by parameterizing the gamma distribution using the performance score (mean) and the square root of the posterior variance (standard deviation) to calculate alpha and beta. In this example, a proportion that is preventable is determined as the area of the posterior distribution worse than the national benchmark.

The method 300 uses at 312, the proportion preventable on the third data set for each measure to calculate the overall preventability score (PS). As an example, a preventability score may be computed as a weighted average of the proportion preventable across each measure, where the weight equals the number of predicted adverse events for each measure. Keeping with the above-example, predicted adverse events are determined as a function of a performance score*number of discharges in the population at risk.

Reference Indicators:

Referring now to FIG. 4, a method 400 illustrates an exemplary approach to generating the quality indicators utilized in the methods 200 and 300 described more fully herein. As such, the method 400 (or select steps thereof) may be a preliminary process for performing the methods 200, 300.

The method 400 obtains at 402, the Agency for Healthcare Research and Quality (AHRQ) quality indicator (QI) software (SAS, Version 4.5) from http://www.qualityindicators.ahrq.gov. This publically available software has parameters embedded therein based upon a national model. By way of illustration, and not by way of limitation, the AHRQ has developed health care decision-making and research tools in the form of software that can be used to identify quality of care events that might need further study. The software programs apply the AHRQ Quality Indicators (QIs) to a data set to assist quality improvement efforts in acute care hospital settings. The software also provides the signal variance utilized at 214 of FIG. 2.

The method 400 also obtains at 402, a reference indicator set of quality indicators. These quality indicators include measures that can be used to highlight potential quality concerns, identify areas that need further study and investigation, and track changes over time. The reference set may be derived for instance, from the obtained software. In an illustrative example, the measures comprise Inpatient Quality Indicators (IQI), Patient Safety Indicators (PSI) and Pediatric Quality Indicators (PDI). Regardless, the reference indicator set will comprise data that is relatively old, e.g., a few years behind the current year, and may span a single year (e.g., 2010), or other relevant time frame.

As noted above with reference to FIGS. 2 and 3, a first data set is utilized to compute a preventability score. In this regard, the method obtains at 404, a first data set that comprises at least one state-wide inpatient database, e.g., a State Inpatient Database (SID). The information collected into each SID is likely to include information concerning community hospitals located within the corresponding state, as well as POA data. By way of example, the SID data for one or more states can be obtained from HCUP at http://www.hcup-us.ahrq.gov. In an exemplary implementation, the SID data is collected over a period of years (e.g., 2008-2011) that span the date range comprehended by the reference indicator set at 402 (e.g., 2010).

Also as noted above with reference to FIGS. 2 and 3, a second data set is also utilized to compute a preventability score. The method obtains at 406, a second data set, e.g., the Nationwide Inpatient Sample (NIS). In a practical implementation, the obtained sample comprises a sample of community hospitals (e.g., a 20% sample of community hospitals) spanning a data range (e.g., 2008-2011). In this regard, POA data is unlikely to be available from the national inpatient sample obtained at 406. The NIS may be obtained from HCUP, e.g., at http://www.hcup-us.ahrq.gov.

As noted yet further above with reference to FIGS. 2 and 3, a third data set is utilized to compute a preventability score. Accordingly, the method obtains at 408, a third data set, designated a Hospital Association (HA) Data set. In an illustrative implementation, the third data set may comprise data collected from community hospitals, which may include data from in-state hospitals, out-of-state hospitals, or a combination thereof. The third data set may include POA data. Moreover, the third data set may comprise data that spans a wider date range than the first data set and/or second data set. For instance, the third data set may include data that spans the same date range as the SID data set and/or NIS data set. The third data set HA may also include data that is more recent than the second data set. For instance, the third data set HA may be logically conceptualized as data in the date range (2008-2011) and data in the date range (2012-2013).

In this regard, there is an inherent delay in accessing SID data and NIS data (first and second data sets) due to the processing delays in collecting and aggregating the data. However, it may be more time efficient to obtain data from data aggregators such as hospital associations or directly from hospitals themselves.

As such, the NIS covers a national data sample, but does not include POA data. The SID data includes POA data, but lags the current period by 18-24 months or longer. The HA data includes POA data, and is more up-to-date compared to SID data. However, the HA data is a smaller data set.

The method 400 maps at 410 data elements and data values from the first data set (e.g., SID data elements and data values) to a software data dictionary, e.g., an AHRQ QI Software data dictionary. The method 400 also maps at 412 data elements and data values from the second data set (e.g., NIS data elements and data values) to the software data dictionary. Still further, the method maps at 214, data elements and data values from the third data set (e.g., HA hospital association data elements and data values) to the software data dictionary.

The method 400 evaluates at 416, the SID data set against the reference data set of quality indicators obtained at 402. The evaluation at 416 calculates an observed present on admission (P) for each discharge and measure in the SID data set. The evaluation at 416 also calculates an expected present on admission (E[P|X]) for each discharge and measure in the SID data set.

The method 400 evaluates at 418, the NIS data set against the reference data set of quality indicators obtained at 402. The evaluation at 418 calculates an observed outcome of interest (Y) for each discharge and measure of the NIS data set. The evaluation at 418 also calculates an expected outcome of interest (E[Y|X]) for each discharge and measure of the NIS data set. The evaluation at 418 further calculates an expected present on admission (E[P|X]) value for each discharge and measure of the NIS data set.

The method 400 evaluates at 420, the HA data set against the reference data set of quality indicators obtained at 402. The evaluation at 420 calculates an observed outcome of interest (Y) for each discharge and measure. The evaluation at 420 also calculates an observed present on admission (P) value for each discharge and measure. The evaluation at 420 still further calculates an expected outcome of interest (E[Y|X]) for each discharge and measure of the HA data set.

A table illustrating a complete non-limiting, yet exemplary method combining FIGS. 2-4 is illustrated below. As illustrated, steps 1-10 are represented in FIG. 4, steps 11-16, are illustrated in FIGS. 2 and 17-21 are illustrated in FIG. 3.

| | | | | | | | Hospital | Hospital |
|---|---|---|---|---|---|---|---|---|
| | | | Ref | Nat'l | Nat'l | | Ass'n | Ass'n |
| | | Ref | Pop | Data | Data | | Data | Data |
| | | Pop | (2008- | (2008- | (2012- | POA | (2008- | (2012- |
| # | Process Step | (2010) | 2011) | 2011) | 2013) | Data | 2011) | 2013) |
| 1 | Obtain the AHRQ QI | X | | | | | | |
| | Software (SAS, Version | | | | | | | |
| | 4.5) from | | | | | | | |
| | http://www.qualityindic | | | | | | | |
| | ators.ahrq.gov. | | | | | | | |
| | Inpatient | | | | | | | |
| | Quality Indicators | | | | | | | |
| | (IQI); 15 measures) | | | | | | | |
| | Patient Safety | | | | | | | |
| | Indicators (PSI); 13 | | | | | | | |
| | measures | | | | | | | |
| | Pediatric | | | | | | | |
| | Quality Indicators | | | | | | | |
| | (PDI); 12 measures | | | | | | | |
| 2 | Obtain the State | | | | | X | X | |
| | Inpatient Databases | | | | | | | |
| | (SID) for selected states | | | | | | | |
| | from http://www.hcup- | | | | | | | |
| | us.ahrq.gov | | | | | | | |
| | 100% of | | | | | | | |
| | community | | | | | | | |
| | hospitals located in | | | | | | | |
| | state | | | | | | | |
| | Present on | | | | | | | |
| | admission (POA) | | | | | | | |
| | data available | | | | | | | |
| | States: CA, | | | | | | | |
| | CO, IA, MD, VT | | | | | | | |
| 3 | Obtain the Nationwide | | | X | | | | |
| | Inpatient Sample (NIS) | | | | | | | |
| | from http://www.hcup- | | | | | | | |
| | us.ahrq.gov | | | | | | | |
| | 20% sample of | | | | | | | |
| | community | | | | | | | |
| | hospitals | | | | | | | |
| 4 | Obtain Hospital | | | | | X | X | X |
| | Association (HA) Data | | | | | | | |
| | 100% of | | | | | | | |
| | community | | | | | | | |
| | hospitals located in | | | | | | | |
| | state (some out-of- | | | | | | | |
| | state members) | | | | | | | |
| | Present on | | | | | | | |
| | admission (POA) | | | | | | | |
| | data available | | | | | | | |
| | States: OH | | | | | | | |
| 5 | Map the SID data | | | | | X | X | |
| | elements and data | | | | | | | |
| | values to the AHRQ QI | | | | | | | |
| | Software data | | | | | | | |
| | dictionary | | | | | | | |
| 6 | Map the NIS data | | | X | | | | |
| | elements and data | | | | | | | |
| | values to the AHRQ QI | | | | | | | |
| | Software data | | | | | | | |
| | dictionary | | | | | | | |
| 7 | Map the HA data | | | | | X | X | X |
| | elements and data | | | | | | | |
| | values to the AHRQ QI | | | | | | | |
| | Software data | | | | | | | |
| | dictionary | | | | | | | |
| 8 | Run the AHRQ QI | X | | | | X | X | |
| | Software on the SID | | | | | | | |
| | P1: Calculates | | | | | | | |
| | the observed | | | | | | | |
| | present on | | | | | | | |
| | admission (P) for | | | | | | | |
| | each discharge and | | | | | | | |
| | measure | | | | | | | |
| | P3: Calculates | | | | | | | |
| | the expected | | | | | | | |
| | present on | | | | | | | |
| | admission (E[P\|X]) | | | | | | | |
| | for each discharge | | | | | | | |
| | and measure | | | | | | | |
| 9 | Run the AHRQ QI | X | | X | | | | |
| | Software on the NIS | | | | | | | |
| | P1: Calculates | | | | | | | |
| | the observed | | | | | | | |
| | outcome of interest | | | | | | | |
| | (Y) for each | | | | | | | |
| | discharge and | | | | | | | |
| | measure | | | | | | | |
| | P3: Calculates | | | | | | | |
| | the expected | | | | | | | |
| | outcome of interest | | | | | | | |
| | (E[Y\|X]) for each | | | | | | | |
| | discharge and | | | | | | | |
| | measure | | | | | | | |
| | P3: Calculates | | | | | | | |
| | the expected | | | | | | | |
| | present on | | | | | | | |
| | admission (E[P\|X]) | | | | | | | |
| | for each discharge | | | | | | | |
| | and measure | | | | | | | |
| 10 | Run the AHRQ QI | X | | | | X | X | X |
| | Software on the HA | | | | | | | |
| | P1: Calculates | | | | | | | |
| | the observed | | | | | | | |
| | outcome of interest | | | | | | | |
| | (Y) for each | | | | | | | |
| | discharge and | | | | | | | |
| | measure | | | | | | | |
| | P1: Calculates | | | | | | | |
| | the observed | | | | | | | |
| | present on | | | | | | | |
| | admission (P) for | | | | | | | |
| | each discharge and | | | | | | | |
| | measure | | | | | | | |
| | P3: Calculates | | | | | | | |
| | the expected | | | | | | | |
| | outcome of interest | | | | | | | |
| | (E[Y\|X]) for each | | | | | | | |
| | discharge and | | | | | | | |
| | measure | | | | | | | |
| 11 | Recalibrate the | X | X | | | X | X | |
| | expected present on | | | | | | | |
| | admission on the SID: | | | | | | | |
| | the “SID P | | | | | | | |
| | Recalibration Factor” | | | | | | | |
| | So the overall | | | | | | | |
| | observed rate (P) | | | | | | | |
| | equals the overall | | | | | | | |
| | expected rate | | | | | | | |
| | (E[P\|X]) for each | | | | | | | |
| | measure | | | | | | | |
| 12 | Use the “SID P | | | X | | X | | |
| | Recalibration Factor” to | | | | | | | |
| | re-calculate expected | | | | | | | |
| | present on admission on | | | | | | | |
| | the NIS: | | | | | | | |
| | P3: Re- | | | | | | | |
| | calculate the | | | | | | | |
| | expected present on | | | | | | | |
| | admission (E[P\|X]) | | | | | | | |
| | for each discharge | | | | | | | |
| | and measure | | | | | | | |
| 13 | Use the re-calculated | X | X | X | | X | | |
| | expected present on | | | | | | | |
| | admission on the NIS to | | | | | | | |
| | re-calculate the | | | | | | | |
| | observed and expected | | | | | | | |
| | outcome of interest on | | | | | | | |
| | the NIS | | | | | | | |
| | P1: Re- | | | | | | | |
| | calculate the | | | | | | | |
| | observed outcome | | | | | | | |
| | of interest (Y, P = 0) | | | | | | | |
| | for each discharge | | | | | | | |
| | and measure | | | | | | | |
| | P3: Re- | | | | | | | |
| | calculate the | | | | | | | |
| | expected outcome | | | | | | | |
| | of interest (E[Y, | | | | | | | |
| | P = 0\|X]) for each | | | | | | | |
| | discharge and | | | | | | | |
| | measure | | | | | | | |
| 14 | Use the re-calculated | X | X | X | X | X | | |
| | observed and expected | | | | | | | |
| | outcome of interest on | | | | | | | |
| | the NIS to forecast the | | | | | | | |
| | observed and expected | | | | | | | |
| | outcome of interest | | | | | | | |
| | Forecast using | | | | | | | |
| | a linear trend of the | | | | | | | |
| | observed-to- | | | | | | | |
| | expected ratio for | | | | | | | |
| | each hospital with a | | | | | | | |
| | seasonally | | | | | | | |
| | (quarterly) effect | | | | | | | |
| 15 | Use the re-calculated | X | X | X | X | X | | |
| | observed and expected | | | | | | | |
| | outcome of interest on | | | | | | | |
| | the NIS and the | | | | | | | |
| | forecasted observed and | | | | | | | |
| | expected outcome of | | | | | | | |
| | interest on the NIS to | | | | | | | |
| | calculate the overall | | | | | | | |
| | observed-to-expected | | | | | | | |
| | ratio and the reference | | | | | | | |
| | population rate (K) for | | | | | | | |
| | each measure | | | | | | | |
| 16 | Use the signal variance | X | X | X | X | X | X | X |
| | from Version 4.5 and | | | | | | | |
| | the reference | | | | | | | |
| | population rate on the | | | | | | | |
| | NIS to calculate the | | | | | | | |
| | “National Benchmark” | | | | | | | |
| | for each measure | | | | | | | |
| | National Benchmark = | | | | | | | |
| | 80^{th} percentile in the | | | | | | | |
| | performance score | | | | | | | |
| | distribution | | | | | | | |
| 17 | Use the re-calculated | X | X | X | X | X | X | X |
| | expected outcome of | | | | | | | |
| | interest on the NIS and | | | | | | | |
| | the forecasted expected | | | | | | | |
| | outcome of interest on | | | | | | | |
| | the NIS to re-calculate | | | | | | | |
| | the expected outcome | | | | | | | |
| | of interest on the HA | | | | | | | |
| | P3: Re- | | | | | | | |
| | calculate the | | | | | | | |
| | expected outcome | | | | | | | |
| | of interest (E[Y, | | | | | | | |
| | P = 0\|X]) for each | | | | | | | |
| | discharge and | | | | | | | |
| | measure | | | | | | | |
| 18 | Use the observed | X | X | X | X | X | X | X |
| | outcome of interest on | | | | | | | |
| | the HA, the re- | | | | | | | |
| | calculated expected | | | | | | | |
| | outcome of interest on | | | | | | | |
| | the HA and the | | | | | | | |
| | reference population | | | | | | | |
| | rate from the NIS to | | | | | | | |
| | calculate the risk- | | | | | | | |
| | adjusted rate on the HA | | | | | | | |
| | and the noise variance | | | | | | | |
| | on the HA for each | | | | | | | |
| | measure | | | | | | | |
| | Risk-adjusted | | | | | | | |
| | rate on HA = | | | | | | | |
| | (observed rate on | | | | | | | |
| | HA/expected rate | | | | | | | |
| | on HA) * reference | | | | | | | |
| | population rate on | | | | | | | |
| | NIS | | | | | | | |
| | Noise variance | | | | | | | |
| | on HA = | | | | | | | |
| | Variance(risk- | | | | | | | |
| | adjusted rate on | | | | | | | |
| | HA) | | | | | | | |
| 19 | Use the risk-adjusted | X | X | X | X | X | X | X |
| | rate on the HA, the | | | | | | | |
| | noise variance on the | | | | | | | |
| | HA and the signal | | | | | | | |
| | variance from Version | | | | | | | |
| | 4.5 to calculate the | | | | | | | |
| | performance score on | | | | | | | |
| | the HA and the | | | | | | | |
| | “posterior variance” on | | | | | | | |
| | the performance score | | | | | | | |
| | on the HA for each | | | | | | | |
| | measure | | | | | | | |
| | Reliability- | | | | | | | |
| | weight (W) = | | | | | | | |
| | (signal variance/ | | | | | | | |
| | (noise variance on | | | | | | | |
| | HA + signal | | | | | | | |
| | variance)) | | | | | | | |
| | Performance | | | | | | | |
| | score = risk- | | | | | | | |
| | adjusted rate on HA | | | | | | | |
| | * W + reference | | | | | | | |
| | population rate on | | | | | | | |
| | NIS * (1-W) | | | | | | | |
| | Posterior | | | | | | | |
| | variance = signal | | | | | | | |
| | variance * (1-W) | | | | | | | |
| 20 | Use the national | X | X | X | X | X | X | X |
| | benchmark, the | | | | | | | |
| | performance score on | | | | | | | |
| | the HA and posterior | | | | | | | |
| | variance on the | | | | | | | |
| | performance score on | | | | | | | |
| | the HA to calculate the | | | | | | | |
| | “proportion | | | | | | | |
| | preventable” on the HA | | | | | | | |
| | for each measure | | | | | | | |
| | Posterior | | | | | | | |
| | distribution = | | | | | | | |
| | parameterize the | | | | | | | |
| | gamma distribution | | | | | | | |
| | using the | | | | | | | |
| | performance score | | | | | | | |
| | (mean) and the | | | | | | | |
| | square root of the | | | | | | | |
| | posterior variance | | | | | | | |
| | (standard deviation) | | | | | | | |
| | to calculate alpha | | | | | | | |
| | and beta | | | | | | | |
| | Proportion | | | | | | | |
| | preventable = the | | | | | | | |
| | area of the posterior | | | | | | | |
| | distribution worse | | | | | | | |
| | than the national | | | | | | | |
| | benchmark | | | | | | | |
| 21 | Use the proportion | X | X | X | X | X | X | X |
| | preventable on the HA | | | | | | | |
| | for each measure to | | | | | | | |
| | calculate the overall | | | | | | | |
| | preventability score | | | | | | | |
| | (PS) | | | | | | | |
| | Preventability | | | | | | | |
| | score = weighted | | | | | | | |
| | average of the | | | | | | | |
| | proportion | | | | | | | |
| | preventable across | | | | | | | |
| | each measure, | | | | | | | |
| | where the weight | | | | | | | |
| | equals the number | | | | | | | |
| | of predicted | | | | | | | |
| | adverse events for | | | | | | | |
| | each measure | | | | | | | |
| | Predicted | | | | | | | |
| | adverse events = | | | | | | | |
| | performance score | | | | | | | |
| | * number of | | | | | | | |
| | discharges in the | | | | | | | |
| | population at risk | | | | | | | |

Quality Indicator:

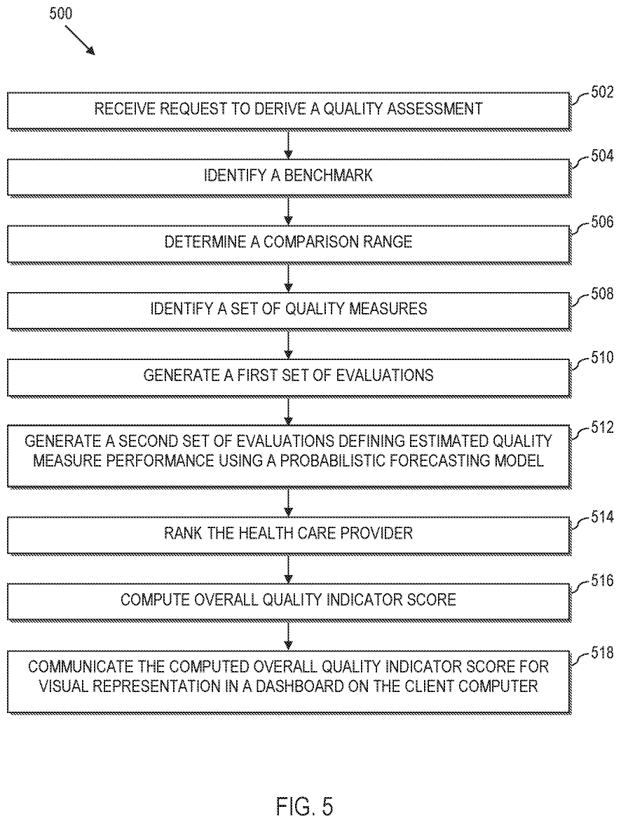

Referring now to FIG. 5, a method 500 is illustrated for providing decision support to a health care provider according to aspects of the present disclosure. More particularly, the method 500 can be implemented by a server interacting with a client computer to display information in a dashboard view.

The method 500 is performed by receiving at 502, a request from a client computer to derive a quality assessment associated with a health care provider of interest, where the quality assessment populates a dashboard on the client computer. By way of example, a user may issue a request by virtue of using a client computer, e.g., a processing device 102 of FIG. 1, to log into the decision support server 106 of FIG. 1. The decision support server 106 receives the request and utilizes the processing engine 112 to derive a quality assessment for the user.

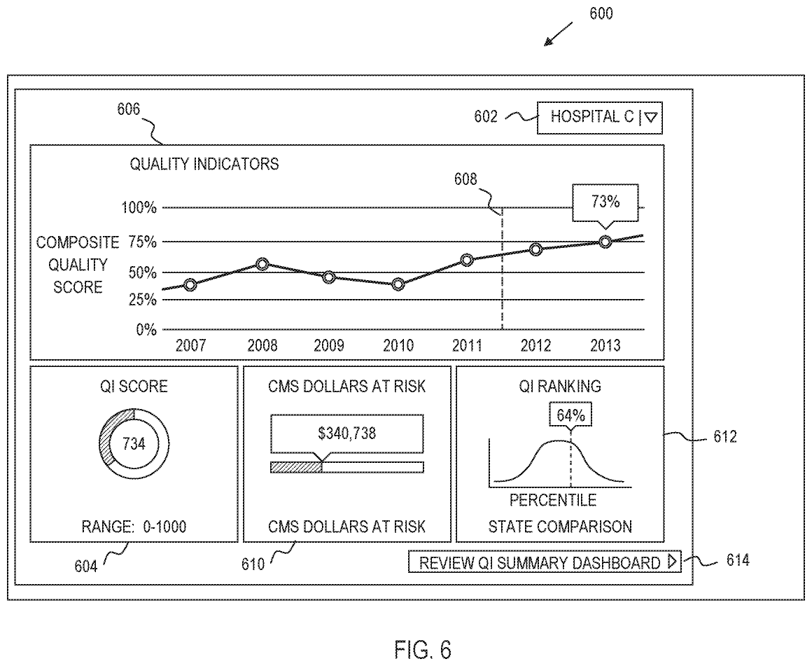

As will be described with reference to FIGS. 6-9, the quality assessment may be implemented as a series of dashboards that the user can dynamically interact with in order to assess various health care metrics. In this illustrative example, since the user must log into the decision support system, the data is limited to a health care provider of interest, e.g., a health care provider has authorized the user. The health care provider of interest may include a hospital, clinic, treatment facility, rehabilitation center, etc. As another example, the health care provider of interest may comprise an association, e.g., a hospital membership organization. In this example, health care providers may be organized in a hierarchy where a user, e.g., an administrator, may oversee multiple different hospitals. Here, the user can use the dashboards to analyze data at the association level, or the user can “zoom” into dashboard views that provide indicators for the performance of the individual represented hospitals.

The method 500 further comprises identifying, at 504, a benchmark that is associated with the quality indicator, where the benchmark defines at least one entity to compare against the health care provider of interest. For instance, the benchmark may default or otherwise be restricted to a national average benchmark. In other implementations, the benchmark may be user-definable, e.g., using a dropdown menu to select between national and state level views, etc. Moreover, the benchmarks need not be geographically limiting. Moreover, the benchmark may be the health care provider of interest itself, e.g., as measured at a previous point in time. As additional examples, the benchmarks may be based upon patient population size, whether the hospital is rural, whether the hospital is member in a particular hospital system, whether the hospital is a teaching hospital, etc.

The method may also comprise determining, at 506, a comparison range over which data from the data source is to be analyzed for deriving the quality indicator. For instance, the comparison range may be specified in years, year to date, quarterly, etc. Again, the range may be automatically fixed by the process, or user adjustable.

The method still further comprises identifying, at 508, a set of quality measures that each assesses a different aspect of health care, e.g., as described with reference to 202, 402 of FIGS. 2 and 4. By way of example, the quality measures may be defined by government agencies, such as the Agency for Healthcare Research & Quality (AHRQ), Centers for Medicare & Medicaid Services (CMS), and Patient-Centered Outcomes Research Institute (PCORI). In this regard, the quality measures report how well the health care provider of interest provides care for patients undergoing medical treatment/procedures, or for patients with a particular medical condition. In this manner, quality measures can assess aspects of health care structure, e.g., types and availability of services), outcomes (e.g., infection rate, mortality, length of stay, etc.), processes (e.g., giving an antibiotic before or after a procedure). In further illustrative examples, custom quality measures can be defined. Moreover, complex quality measures can be constructed from existing quality measures. In certain example implementations, the quality measures may be fixed by the process. In alternative implementations, the user may be able to filter or otherwise select quality measures of interest.

As will be described in greater detail herein, the method 500 may be used to perform evaluations based upon a time frame that requires some data points to be based upon forecast values.

The method 500 thus comprises generating, at 510, a first set of quality measure performance evaluations by evaluating the set of quality measures against a subset of the underlying medical data in the data source that has been filtered by the range (e.g., filtered by year to date, a user select quarter, a range of years, etc.). A first set of quality measure performance evaluations is computed using available data, e.g., based upon a model or set of models such as the national Quality Indicator models, models from other private or government agencies, nationally representative administrative data such as HCUP, and optionally, other available data, such as from an aggregator, from the health care provider of interest, etc. In this regard, the quality indicator models developed for the quality measures may be made utilized, e.g., such as where the quality measures are defined by government agencies, such as the AHRQ, Center for Medicare and Medicaid Services (CMS), and PCORI.

The method 500 further comprises generating, at 512, a second set of quality measure performance evaluations defining an estimated quality measure performance using a probabilistic forecasting model (or models) to evaluate the set of quality measures for the healthcare provider of interest (e.g., as computed at 208, 210 of FIG. 2). By way of example, the probabilistic forecasting model (or models) can be generated using logistic regression models to model adverse events based upon average trends across the nation. In this regard, regression coefficients can be utilized to adjust factors associated with adverse events of interest.

For instance, government models such as generated by the AHRQ, CMS, and PCORI, are likely to be two-years old (or older) due to inherent lags in current methods of data gathering. This leaves a significant blind spot in the assessment of quality of a health care provider interested in current trends and indicator values. However, this gap is closed by the probabilistic forecasting model (or models) of the decision support system herein. Here, the probabilistic forecasting models can be updated quarterly, or on other basis, such as where the health care provider has sufficient data to present to the decision support system.

In certain embodiments, the method 500 performs ranking, at 514, of the health care provider of interest for each measure in the set of quality measures. The ranking may be based upon a user-selected comparison group, e.g., state-wide ranking, national ranking, etc.

In certain embodiments, the method 500 further comprises computing, at 516, a single, overall quality indicator score, e.g., based upon the preventability score described with reference to FIGS. 2-4. For instance, a score (such as the score at 604 of FIG. 6) can be computed by looking back at the last four previous quarters. In this example, the healthcare provider score may be computed based upon forecast data only. As another example, a score can be averaged out across a longer period of time that comprehends both forecast and measureable data.

Also, the method 500 comprises communicating, at 518, the computed overall quality indicator score for visual representation in the dashboard on the client computer.

Aspects of the present disclosure can thus compare deviations from a national curve as a function of what a health care provider is able to achieve based upon the case mix of the health care provider at a prescribed period of time. That is, scores can be computed that reflect how a given health care provider is performing with regard to their case mix in view of a national average. For instance, a hospital may be improving, but at a rate slower than a national average. Thus, the hospital rating is adjusted for this.

The User Interface Dashboard:

Referring to FIG. 6, a dashboard 600 is illustrated. The dashboard, or components thereof, may be generated as a result of executing the methods of FIGS. 2-5 or combinations thereof. The dashboard 600 may also and/or alternatively be implemented using the system of FIG. 1.

The dashboard 600 is the entry screen into the dashboard software product, which will provide secure access to the available measures and metrics for authorized users from a particular hospital. In this example, the health care provider of interest is “Hospital C”, which represents a simulated small rural community hospital, as selected by the dropdown menu selection 602. The dashboard 600 demonstrates an overall composite quality score and how it changes/trends over time for the Hospital C, along with their estimated quality indicator (QI) composite score for the current calendar year (e.g., as computed using the methods described with reference to FIGS. 2-4). For instance, in the illustrative example, the decision support system computes a quality indicator score for Hospital C of 734. The computed score represents a score normalized as a number in the range of 0-1000. This is illustrated at 604 as a numeric value circled by a ring that is shaded to also visually depict the score.

The user of the dashboard 600 sends a request from a client computer to a decision support server to derive a quality assessment associated with a health care provider of interest, where the quality assessment populates a dashboard on the client computer. The user will have previously been required to log into the system using secure login credentials (not shown).

The user may have the option to specify a user-selected dashboard benchmark (i.e., comparison group), where the dashboard benchmark defines at least one entity to compare against the health care provider of interest. In this regard, the dashboard benchmark can be user-adjusted or set as a default or non-adjustable parameter, e.g., to a national comparison. The user may also optionally determine a comparison range over which data from the data source is to be analyzed for deriving the quality indicator. The range may be a metric such as year to date, current quarter, etc. The initial range may be set by default, or the range may be user-specified.

The decision support system further computes a single, overall quality indicator score, e.g., based upon a comparison of a first data set, a second data set, and the benchmark as described in greater detail herein. The computed overall quality indicator score is communicated to the client computer for visual representation in the dashboard.

The dashboard 600 also provides a quality indicator trend over time, in the form of a chronological trend graph 606. For instance, the trend over time may be determined by computing a set of instances of the quality indicator score for the health care provider of interest (e.g., Hospital C in this example), where each instance of the quality indicator score is based upon a different chronological reference. By way of example, the chronological trend graph 606 is illustrated as a time series where a quality indicator score is computed for Hospital C on a yearly basis. Notably, the decision support system communicates the computed set of instances of the quality indicator score for visual representation in the dashboard 600 on the client computer as a chronological trend graph with year on the abscissa and composite quality score in percentage on the ordinate as computed across a national average.

The decision support system further communicates a delineation 608 for display on the chronological trend graph 606. The delineation 608 separates a first group of instances of the quality indicator score that are computed by evaluating the set of quality measures against the underlying medical data in the data source and a second group of instances of the quality indicator score that are estimated by evaluating the set of quality measures for the healthcare provider of interest using the probabilistic forecasting model herein. For instance, as illustrated, the first group of quality indicator scores is the scores computed for years 2007, 2008, 2009, 2010, and 2011. It may be possible for the decision support system to compute these scores based upon the models and data provided in the data sources (e.g., aggregated data sources). The second group of quality indicators is the scores computed for years 2012 and 2013. Here, there is no data (or limited data) available at the national level or otherwise in the aggregated data sources 114. However, the decision support system utilizes the probabilistic forecasting model(s) to evaluate the set of quality measures for Hospital C.

In an illustrative example, values in the time-series graph that are to the left of the vertical dashed-line (2011 and prior) are based on models developed by AHRQ and CMS (or their contractors) that were applied to the National HCUP Data; whereas the values to the right of the dashed-line (2012 and 2013) are based on the predictive models herein to extend inferences beyond the availability of national data and national models.

The decision support system further computes, at 610, an estimate of reimbursable dollars at risk. For instance, the reimbursable dollars at risk may be computed by integrating the estimated quality measure performance for the health care provider of interest (e.g., estimates computed for Hospital C for the current calendar year using the probabilistic forecasting model) against reimbursement policies, and a fraction of the patient population cared for by the health care provider of interest (Hospital C in this example) that are supported by associated reimbursement programs. The decision support system further communicates the computed estimate of reimbursable dollars at risk for visual representation in the dashboard 600 on the client computer. For instance, in the illustrated example, reimbursable dollars for CMS Dollars at Risk are displayed at 610 as a numeric dollar amount and on a visual meter.

As such, the metric at 610 informs the user of the money at “risk” based on reimbursement policies (e.g., CMS reimbursement policies in this example), either as losses or profit, based on current and predicted quality score.

The decision support system may further communicate a histogram 612 for visual representation in the dashboard 600 on the client computer. The histogram in the lower right-hand corner of the entry screen demonstrates where Hospital C's overall composite score ranks among a chosen comparison population (in this example, the dashboard benchmark/comparison group represents all hospitals across the state in which Hospital C is located). The histogram 612 visually depicts an empirical distribution of the quality indicator score across a user-selected dashboard benchmark (e.g., the state that Hospital C is located) with an indication of the computed overall quality indicator score for the health care provider of interest within the histogram (e.g., Hospital C is illustrated with a quality indicator score ranking them in the 64% percentile across their state).

Note that if Hospital C was a member of a larger hospital system, then a user at the Hospital System management level might be authorized to view multiple other hospitals within the same system. The screen is designed to allow this type of user to use a drop-down menu to select other hospitals within the same system by depressing the down-arrow in the top right-hand part of the screen (next to Hospital C), e.g., by selecting a different hospital using the menu option at 602.

To get to the next screen in the software tool, the user can click on the arrow at the bottom next to the phrase “Review QI Summary Dashboard” at the navigation option 614.

Quality Indicator Summary Dashboard:

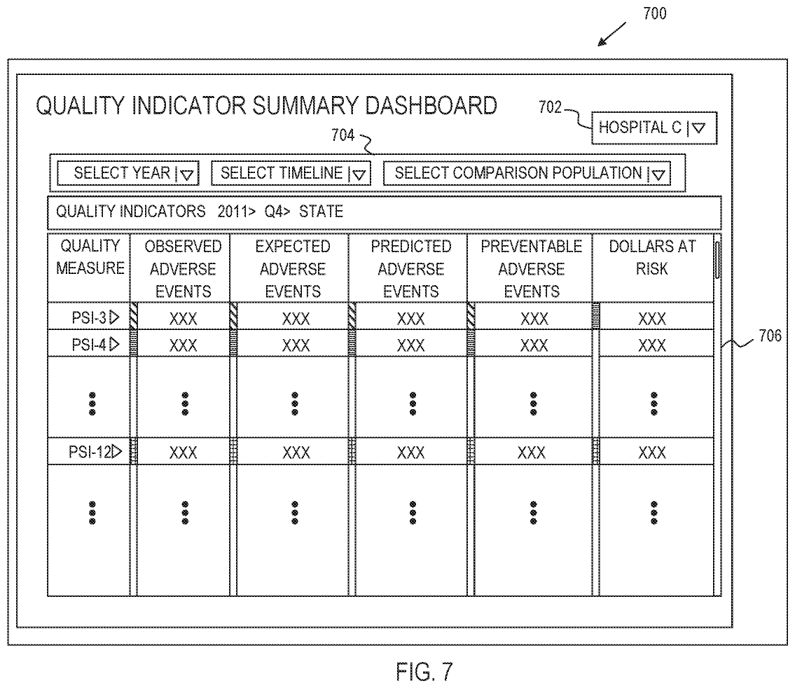

Referring to FIG. 7, a Quality Indicator Summary dashboard 700 is illustrated. The quality indicator summary dashboard 700 is a dashboard page accessed by selecting the navigation option 614 of FIG. 6. The Quality Indicator Summary Dashboard 700 represents the main navigation page for the software tool. The user-interface of the dashboard 700 includes a health care provider selection box 702, which is analogous to the corresponding box providing the menu option at 602 described with reference to FIG. 6.

The user interface of the dashboard 700 further provides inputs 704 for the user to dynamically custom filter a table of data that is displayed in a main dashboard view. For instance, in the illustrative example, inputs 704 are provided for the user to enter a year (start of time for data collection to present), a timeline (e.g., first quarter—Q1, second quarter—Q2, third quarter—Q3, fourth quarter—Q4) and a comparison population (a dashboard benchmark such as national, state, regional, rural hospitals, teaching hospitals, hospitals that are members of a member association, etc.). For instance, in the illustrated example, the user-selected 2011, Quarter 4, and a comparison population across the State associated with Hospital C. In this regard, the drop down menus may be customized to each hospital, e.g., based upon dashboard benchmarks that are meaningful to the health care provider (Hospital C for instance). In response to receiving user selections for the year, time frame and comparison population, the processor computes the table 706.

The table 706 includes a listing of the set of quality measures in a Quality Measure field. An observed number of adverse events is presented in the Observed Adverse Events column. A number of expected adverse events is presented in the Expected Adverse Events column. A number of predicted adverse events appear in a Predicted Adverse events column. A number of preventable adverse events is presented in the Preventable Adverse Events column. Here, the preventable adverse events are measured as of the designated percentile (e.g., 80th percentile). This measures “how many” adverse events would have occurred if the health care provider were operating at the designated percentile, e.g., 80% percentile. Other percentiles could alternatively have been used. An estimated amount of reimbursable dollars at risk for the health care provider of interest is presented in a Dollars at Risk field. Note that not all measures need be impacted by reimbursement policy.

In other implementations, additional and/or alternative fields may be presented. For instance, additional/alternative columns may include a computed rank of the health care provider of interest for each quality measure in the set of quality measures (listed in the Quality measure field) may be provided in a Rank field. An estimated number of adverse events for the health care provider of interest may be provided in a Preventable Events field. An estimated preventable cost for the health care provider of interest may be provided in a Preventable Cost field. An estimated number of preventable days of care for the health care provider of interest may be provided in a Preventable Days field. The table 706, once generated, is communicated from the server to the client computer for visual representation in the dashboard 700.

The user can dynamically interact with the table 706. For instance, the decision support system can receive a user-selection of sort order, such as by clicking on any one of the fields to dynamically sort the table based upon a user-selected one of fields of the table. In this regard, the decision support system communicates the sorted table for visual representation in the dashboard 700 on the client computer. The user can also vary the data by dynamically interacting with the inputs 704 to alter the filter criteria.

There is a color bar next to each of quality measure metrics for each row (green signifies high quality, yellow signifies moderate quality, and red signifies poor-quality—differentiated in FIG. 7 by different cross-hatch) based on how well the hospital is performing compared to the selected dashboard benchmark comparison group.

There is also a scroll bar to the far-right that allows the user to navigate down through the list. For instance, there are over 90 measures currently available through AHRQ based on administrative data, and numerous other measures from CMS and others that will be available through this dashboard.

Similar to the previous screen, if Hospital C was a member of a larger hospital system, then a user at the Hospital System management level might be authorized to view multiple other hospitals within the same system, and can do so by accessing a drop-down menu to select other hospitals within the same system by depressing the down-arrow in the top right-hand part of the screen (next to Hospital C).

Additionally, the user can place the cursor over any particular Quality Measure, and get a description of the measure in a pop-up window.

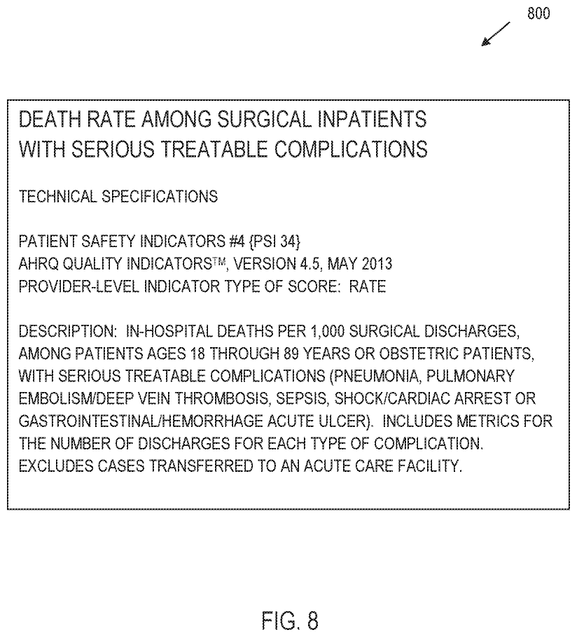

Referring briefly to FIG. 8, if the user mouses-over quality measure PSI-34, a pop up box shows an explanation of the relevant data concerning quality measure PSI-34.

Referring back to FIG. 7, the user can also navigate to the third dashboard screen-type by clicking on the arrow next to any particular quality measure.

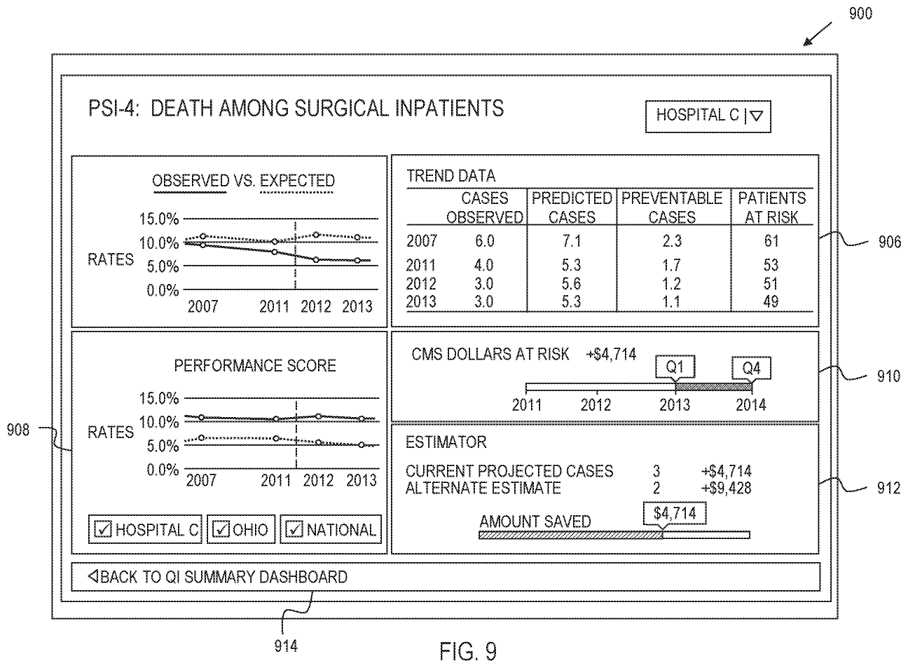

Referring to FIG. 9, a detail dashboard 900 is illustrated. The detail dashboard 900 illustrates the details behind the score computed for a specifically selected quality measure. In general, the decision support system communicates a user-interface (the quality indicator summary dashboard 700) to the client computer. The user-interface includes a health care provider selection box 702, which is analogous to the corresponding box 602 described with reference to FIG. 6.

The dashboard 900 illustrates an example of a detail where the user had clicked on the arrow next to PSI-4 on the Quality Indicator Summary Dashboard. This screen provides detailed information on how the subject health care provider of interest has been performing over time for PSI-4 (which represents Death among Surgical Inpatients for purposes of example).

As the dashboard 900 illustrates, an observed vs. expected chart 904 is provided. More particularly, the decision support system receives a user selection of a select one of the quality measures in the set of quality measures, e.g., from the table listing in the dashboard 700 described with reference to FIG. 7. The decision support system generates a detail page that provides the graph of observed compared to expected rates for the selected quality measure by computing a set of quality measure scores specific to the user-selected quality measure for the health care provider of interest. Each instance of the quality measure score is based upon a different chronological reference and includes an observed value and an expected value. The expected value is based on a case-mix of patients within the hospital of interest.

The decision support system communicates the computed set of quality measure scores for a visual representation in the dashboard 900 on the client computer as a chronological quality measure trend graph that plots the observed values compared to the expected values. The decision support system further communicates a delineation (dashed line between 2011 and 2012) for display on the chronological quality measure trend graph. The delineation is analogous to the delineation 608 of FIG. 6. For instance, the delineation separates a first group of instances of the quality measure scores that are computed by evaluating the user-selected quality measure against the underlying medical data in the data source and a second group of instances of the quality measure scores that are estimated by evaluating the user-selected quality measure for the healthcare provider of interest using the probabilistic forecasting model.

In an illustrative example, the chart 904 illustrates the observed versus expected rates of this adverse event within the subject health care provider of interest. Expected rates are based on the case-mix of patients within the subject health care provider of interest (e.g., the expected rate takes into consideration the distribution of the at-risk population of patients with respect to age, gender, race/ethnicity, and a variety of other factors as specified in the model from AHRQ or CMS).

The decision support system engine further generates on the detail page, a chart 906 of observed compared to expected rates for the selected quality measure. The chart is generated by computing a set of quality measure trends specific to the user-selected quality measure for the health care provider of interest. Each instance of the quality measure trend is based upon a different chronological reference and includes an observed number of cases, an expected number of cases based on a case-mix of patients within the hospital of interest, a number of preventable cases, and a number of patients at risk. Here, a first group of instances of the quality measure trends are computed by evaluating the user-selected quality measure against the underlying medical data in the data source. A second group of instances of the quality measure trends that are estimated by evaluating the user-selected quality measure for the healthcare provider of interest using the probabilistic forecasting model.

In an illustrative example, the Observed vs. Expected graph is the actual trend data, which provides a numerical summary of the number of cases observed, number of predicted cases, number of preventable cases (an estimate that is calculated based on what would be expected from a hospital that is performing well on this particular measure), and the number of patients at risk, for each year observed.

The decision support system also provides a graph of observed compared to expected rates 908 for the selected quality measure. The graph of observed compared to expected rates 908 is generated by computing a set of provider-specific performance scores specific to the user-selected quality measure for the health care provider of interest, where each instance of the provider-specific performance score computed by applying a shrinkage estimator (i.e., the reliability-weight (W) described above) that removes noise in the trend over time for data specific to the health care provider of interest. Thus, the shrinkage estimator corrects for non-systematic unknown source variability.

More specifically, the decision support system computes a set of aggregated performance scores specific to the user-selected quality measure, where each instance of the aggregated performance score is based upon a different chronological reference and is computed across the underlying data as filtered by the dashboard benchmark. The decision support system further communicates the computed set of provider-specific performance scores and aggregated performance scores for visual representation in the dashboard on the client computer as a chronological performance score trend graph that plots the provider-specific performance scores compared to the aggregated performance scores.

The decision support system also communicates a delineation (illustrated as a dashed vertical line between years 2011 and 2012) for display on the chronological quality measure trend graph. The delineation separates a first group of instances of the performance scores that are computed by evaluating the user-selected quality measure against the underlying medical data in the data source and a second group of instances of the performance scores that are estimated by evaluating the user-selected quality measure for the healthcare provider of interest using the probabilistic forecasting model.

In this regard, the performance of the health care provider of interest is captured using the performance score—which is an observed/expected ratio (captured at 608) that applies a shrinkage estimator (i.e., the reliability-weight (W) described above) that enables the ability to remove some of the noise in the trend over time for hospitals with smaller patient populations. The performance score graphic allows the user to check a box to display a credible-interval around the trend over time—which provides a measure of uncertainty around the estimate. The performance score graphic also allows the user to plot the aggregated performance score for the selected quality measure among a dashboard benchmark comparison population.

Similar to the graph 606 in the dashboard 600—values in the time-series graphs that are to the left of the vertical dashed-line (2011 and prior) are based on available data, e.g., models developed by AHRQ and CMS (or their contractors) that were applied to the National HCUP Data; whereas the values to the right of the dashed-line (2012 and 2013) are based on the models herein that extend inferences beyond the available data using probabilistic forecasting models.

A dollars at risk chart is provided at 910. For instance, if the quality measure selected by the user is tied to a CMS reimbursement policy, then the user can select a time-period for estimating the amount of CMS dollars at-risk based on current estimated performance (in this figure, the time period selected represents the Q1 through Q4 in 2013).

An estimator is provided at 912. The ‘Estimator’ 912 is a simulation tool that displays the number of anticipated adverse events over the defined period of time; and then allows the user to estimate of the amount of additional money that would be either gained or lost if the number of adverse events changes from the estimated value (in this case from 3 to 2).

By way of example, the system can utilize the quality measures to identify and evaluate available data to identify events that happen, the cost of those events, and the number of days of care per event. For each quality measure, the system can identify observed cases. Moreover, expected events can be estimated based upon the case mix, e.g., and a national model. The system can then predict the number of events, e.g., a best estimate that places less emphasis on the current case mix. This is a measurement over time. A shrinkage estimator may be applied to the calculations where helpful for certain data sets. The system can then compare the estimates to a standard, e.g., a preventable number of events if performing at the 80th percentile, e.g., many events would the health care provider have seen compared to how many events the health care provider actually saw.

Similar to the previous screens, if Hospital C was a member of a larger hospital system, then a user at the Hospital System management level might be authorized to view multiple other hospitals within the same system, and can do so by accessing a drop-down menu to select other hospitals within the same system by depressing the down-arrow in the top right-hand part of the screen (next to Hospital C).

The user can also navigate back to the Quality Indicator Summary Dashboard by clicking on the navigation arrow 914 at the bottom.

Example Computer Implementation:

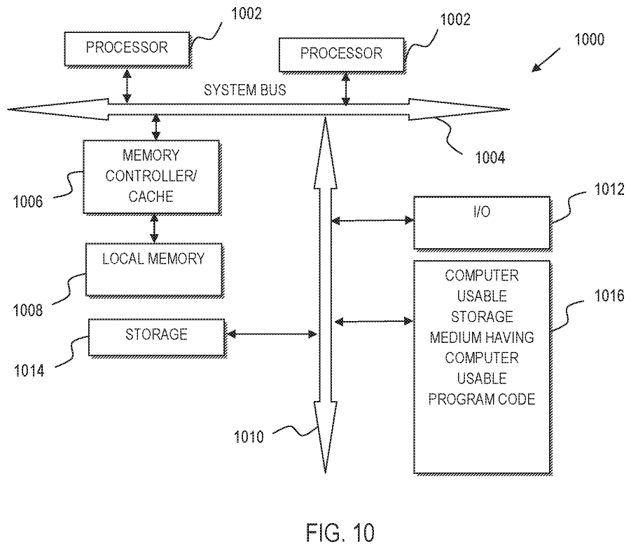

Referring to FIG. 10, a block diagram of a data processing system is depicted in accordance with the present disclosure. Data processing system 1000 may comprise one or more processors 1002 connected to system bus 1004. Also connected to system bus 1004 is memory controller/cache 1006, which provides an interface to local memory 1008. An I/O bus 1010 is connected to the system bus 1004 and provides an interface to I/O devices 1012, such as input output devices (I/O devices), storage, network adapters, graphic adapters, etc.

Also connected to the I/O bus 1010 may be devices such as one or more storage devices 1014 and a computer usable storage medium 1016 having computer usable program code embodied thereon. The computer usable program code may be executed, e.g., by the processor(s) 1002 to implement any aspect of the present disclosure, for example, to implement any aspect of any of the methods, processes and/or system components illustrated in FIGS. 1-9.

The present disclosure may be a system, a method (e.g., machine-executable process), a computer program product, or combination thereof. As such, in certain embodiments, a computer-readable storage medium (or media) includes computer readable program instructions thereon for causing a processor to carry out aspects of the present disclosure. In this regard, any combination of computer-readable medium may be utilized. The computer-readable medium may be a computer readable signal medium, a computer-readable storage medium (computer-readable hardware), or a combination thereof.

More specifically, a computer-readable signal medium is a transitory propagating signal per se. A computer-readable signal medium may include computer readable program code embodied therein, for example, as a propagated data signal in baseband or as part of a carrier wave. Thus, a propagating signal encompasses radio waves or other freely propagating electromagnetic waves. However, a computer-readable signal medium is not hardware.

To the contrary, a computer readable storage medium is a tangible device (hardware) that can retain and store instructions for use by an instruction execution device, e.g., the hardware aspects of the system described with reference to FIG. 10, the hardware aspects of the processing device(s) 102, server 106 of FIG. 1, etc. Thus, a computer readable storage medium, as used herein, is not a transitory signal per se. Exemplary and non-limiting structures for implementing a computer readable storage medium include a portable computer diskette, a hard disk, a random access memory (RAM), Flash memory, a read-only memory (ROM), a portable compact disc read-only memory (CD-ROM), digital video disk (DVD), an optical storage device, a magnetic storage device, or any suitable combination of the foregoing.

Aspects of the present disclosure are described herein with reference to flowchart illustrations and/or block diagrams of methods, apparatus (systems) and computer program products according to embodiments of the disclosure. Each block of the flowchart illustrations and/or block diagrams, and combinations of blocks in the flowchart illustrations and/or block diagrams, can be implemented by computer program instructions, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Each block in the flowchart or block diagrams of the FIGURES herein, may represent a module, segment, or portion of code, which comprises one or more executable instructions for implementing the specified logical function(s). However, the functions noted in the block may occur out of the order noted in the figures. For example, two blocks shown in succession may, in fact, be executed substantially concurrently, or the blocks may sometimes be executed in the reverse order, depending upon the functionality involved.

The terminology used herein is for the purpose of describing particular embodiments only and is not intended to be limiting of the disclosure. As used herein, the singular forms “a,” “an,” and “the” are intended to include the plural forms as well, unless the context clearly indicates otherwise. Further, the terms “comprises” and “comprising,” when used in this specification, specify the presence of stated features, steps, operations, elements, and/or components, but do not preclude the presence or addition of one or more other features, steps, operations, elements, components, and/or groups thereof.

The corresponding structures, materials, acts, and equivalents of any means or step plus function elements in the claims below are intended to include any disclosed structure, material, or act for performing the function in combination with other claimed elements as specifically claimed. The description of the present disclosure has been presented for purposes of illustration and description, but is not intended to be exhaustive or limited to the disclosure in the form disclosed. Many modifications and variations will be apparent to those of ordinary skill in the art without departing from the scope and spirit of the disclosure. The aspects of the disclosure herein were chosen and described in order to best explain the principles of the disclosure and the practical application, and to enable others of ordinary skill in the art to understand the disclosure with various modifications as are suited to the particular use contemplated.

Having thus described the disclosure of the present application in detail and by reference to embodiments thereof, it will be apparent that modifications and variations are possible without departing from the scope of the disclosure defined in the appended claims.

What is claimed is:

1. A machine-executable process comprising:
electronically obtaining, over the Internet, state-wide data from the Healthcare Cost and Utilization Project (HCUP), the state-wide data comprising patient present-on-admission (POA) data, the state-wide data comprising data from a first time period, the first time period comprising multiple prior years;
electronically obtaining, over the Internet, national data without POA data, the national data being obtained from the HCUP, the national data comprising data from a second time period, the second time period comprising a prior year;
electronically obtaining, over the Internet Hospital Association (HA) data, the HA data comprising data from a third time period, the third time period comprising a year that is more recent than the first time period;
determining with machine-executable instructions a first POA value, the first POA value being an observed POA value from the state-wide data;
determining with machine-executable instructions a second POA value, the second POA value being an expected POA value for the state-wide data set;
calibrating with machine-executable instructions the second POA value as a recalibration factor such that an overall observed rate equals an overall expected rate for the state-wide data;
determining with machine-executable instructions a third POA value using the recalibration factor, the third POA value being an expected POA value for the national data;
determining with machine-executable instructions a first outcome-of-interest value and a second outcome-of-interest value using the third POA value, the first outcome-of-interest value being an observed outcome-of-interest value from the national data, the second outcome-of-interest value being an expected outcome-of-interest value for the national data;
forecasting with machine-executable instructions a third outcome-of-interest value and a fourth outcome-of-interest value, the third outcome-of-interest value being a forecasted observed outcome-of-interest value for the national data, the fourth outcome-of-interest value being a forecasted expected outcome-of-interest for the national data, the third outcome-of-interest value and the fourth outcome-of-interest value being forecast using:
the first outcome-of-interest value; and
the second outcome-of-interest value;
determining with machine-executable instructions an overall national observed-to-expected ratio and a national reference population rate using:
the first outcome-of-interest value;
the second outcome-of-interest value;
the third outcome-of-interest value; and
the fourth outcome-of-interest value;
determining with machine-executable instructions a national benchmark using:
a predetermined signal variance; and
the national reference population rate;
determining with machine-executable instructions a fifth outcome-of-interest value, the fifth outcome-of-interest value being an expected outcome-of-interest for the HA data, the fifth outcome-of-interest value being calculated using:
the second outcome-of-interest value; and
the fourth outcome-of-interest value;
determining with machine-executable instructions a sixth outcome-of-interest value, the sixth outcome-of-interest value being an observed outcome-of-interest value from the HA data;
determining with machine-executable instructions a risk-adjusted rate on the HA data and a noise variance on the HA data using:
the fifth outcome-of-interest value;
the sixth outcome-of-interest value; and
the national reference population rate;
determining with machine-executable instructions a performance score on the HA data and a posterior variance on the performance score using:
the risk-adjusted rate on the HA data;
the noise variance on the HA data; and
the predetermined signal variance; and
determining with machine-executable instructions a proportion preventable value on the HA data using:
the national benchmark;
the performance score on the HA data; and
the posterior variance on the performance score.

2. The machine-executable process of claim 1 further comprising:
generating with machine-executable instructions a preventability score (PS) that characterizes a proportion of adverse events that were potentially preventable in accessing a healthcare provider of interest based upon the generated benchmark, the HA data, and quality indicators.

3. The machine-executable process of claim 2, wherein the fifth outcome of interest is determined as (E[Y, P=0|X]) for each discharge and quality measure, wherein P represents an observed POA value, wherein E[P|X] represents an expected POA value, and wherein Y represents an observed outcome-of-interest value.

4. The machine-executable process of claim 2, wherein determining the risk-adjusted rate comprises dividing the sixth outcome-of-interest value by the fifth outcome-of-interest value and multiplying the result of the division by the national reference population rate.

5. The machine-executable process of claim 2 further comprising:
generating with machine-executable instructions a reliability-weight (W) as (the predetermined signal variance/(the noise variance on the HA data+the predetermined signal variance));
generating with machine-executable instructions the performance score as (the risk-adjusted rate on the HA data*W)+(national reference population rate*(1−W)); or
generating with machine-executable instructions the posterior variance as (the signal variance*(1−W)).

6. The machine-executable process of claim 2 further comprising:
determining with machine-executable instructions a posterior distribution by parameterizing a gamma distribution using the performance score and the square root of the posterior variance to calculate alpha and beta.

7. The machine-executable process of claim 2 further comprising:
using the proportion preventable on the HA data for each quality measure to calculate with machine-executable instructions an overall preventability score (PS).

8. The machine-executable process of claim 7, wherein using the proportion preventable comprises:
generating with machine-executable instructions the overall PS as a weighted average of the proportion preventable value across each quality measure, where the weight equals a number of predicted adverse events for each quality measure.

9. The machine-executable process of claim 8 further comprising:
determining with machine-executable instructions predicted adverse events as a function of the PS*number of discharges in the population at risk.

10. The machine-executable process of claim 1 further comprising:
processing, by a processor, the state-wide data by:
applying quality indicators against the state-wide data to generate the first POA value; and
generating the second POA value;
processing, by the processor, the national data by:
generating the first outcome-of-interest value;
generating the second outcome-of-interest value; and
generating the third POA value; and
processing, by the processor, the HA data by:
generating the sixth outcome-of-interest value;
generating a fourth POA value, the fourth POA value being an observed POA value for the HA data; and
generating the fifth outcome-of-interest value.

11. The machine-executable process of claim 1 further comprising obtaining quality indicators, the quality indicators being:
Inpatient Quality Indicators (IQI), Patient Safety Indicators (PSI), or Pediatric Quality Indicators (PDI).

12. The machine-executable process of claim 1 further comprising:
using a linear trend of the observed-to-expected ratio for each healthcare provider with a periodic effect.

13. The machine-executable process of claim 1, wherein calculating the national benchmark comprises specifying the national benchmark as a percentile in a performance score distribution.

14. The machine-executable process of claim 1 further comprising:
obtaining the Agency for Healthcare Research and Quality (AHRQ) quality indicator (QI) software; and
using the obtained software to evaluate, with machine-executable instructions, quality indicators against the state-wide data and the national data.

15. The machine-executable process of claim 14 further comprising:
mapping data elements and data values from state-wide data and the national data to an AHRQ QI Software data dictionary.

16. The machine executable process of claim 1 further comprising:
receiving at a client computer, at least one parameter that sets a scope of a benchmark used for a quality assessment associated with a healthcare provider of interest.

17. The machine executable process of claim 2 further comprising:
presenting to a user an interactive graphical user interface for selecting and viewing measures that affected the computed the PS for a healthcare provider of interest.
